(19) United States
(12) Patent Application Publication
(10) Pub. No.: US 2020/0178799 A1
(43) Pub. Date: Jun. 11, 2020

(54) MULTIPLE MODES OF TRANSCEIVER OPERATION IN ANALYTE MONITORING SYSTEM

(71) Applicant: Senseonics, Incorporated, Germantown, MD (US)

(72) Inventors: Barkha Raisoni, Germantown, MD (US); Shang Zhao, Germantown, MD (US); Steven J. Walters, Ellicott City, MD (US)

(73) Assignee: Senseonics, Incorporated, Germantown, MD (US)

(21) Appl. No.: 16/709,345

(22) Filed: Dec. 10, 2019

Related U.S. Application Data
(60) Provisional application No. 62/777,568, filed on Dec. 10, 2018.

(51) Int. Cl. A61B 5/00; A61B 5/145; A61B 5/1495

(52) U.S. Cl. CPC A61B 5/0004; A61B 5/14503; A61B 5/14532; A61B 2560/0204; A61B 5/742; A61B 5/746; A61B 5/1495; A61B 5/0031

(57) ABSTRACT

An analyte monitoring system may include an analyte sensor, a transceiver, and a display device. The transceiver may be configured to operate in two or more modes of operation. The display device may be configured to communicate with the transceiver. The two or more modes of operation may include a clinical mode in which the transceiver receives first sensor data from the analyte sensor and calculates one or more first analyte levels using at least the received first sensor data but does not convey the one or more first analyte levels to the display device. The two or more modes of operation may include an unblinded mode in which the transceiver receives second sensor data from the analyte sensor, calculates one or more second analyte levels using at least the received second sensor data, and conveys the one or more second analyte levels to the display device.

### CROSS-REFERENCE TO RELATED APPLICATION

The present application claims the benefit of priority to U.S. Provisional Application Ser. No. 62/777,568, filed on Dec. 10, 2018, which is incorporated herein by reference in its entirety.

### BACKGROUND

### Field of Invention

Aspects of the present invention relate generally to systems and methods for analyte monitoring. Specifically, aspects of the present invention may relate to multiple modes of operation for a transceiver of an analyte monitoring system. The multiple modes of operation may include, for example and without limitation, two or more of clinical, unblinded, and blinded modes.

### Discussion of the Background

The prevalence of diabetes mellitus continues to increase in industrialized countries, and projections suggest that this figure will rise to 4.4% of the global population (366 million individuals) by the year 2030. Glycemic control is a key determinant of long-term outcomes in patients with diabetes, and poor glycemic control is associated with retinopathy, nephropathy and an increased risk of myocardial infarction, cerebrovascular accident, and peripheral vascular disease requiring limb amputation. Despite the development of new insulins and other classes of antidiabetic therapy, roughly half of all patients with diabetes do not achieve recommended target hemoglobin Alc (HbAlc) levels<7.0%.

Frequent self-monitoring of blood glucose (SMBG) is necessary to achieve tight glycemic control in patients with diabetes mellitus, particularly for patients that require insulin therapy. However, current blood (finger-stick) glucose tests are burdensome, and patient adherence to the recommended frequency of SMBG decreases substantially over time. Moreover, finger-stick measurements only provide information about a single point in time and do not yield information regarding intraday fluctuations in blood glucose levels that may more closely correlate with some clinical outcomes.

Continuous glucose monitors (CGMs) have been developed in an effort to overcome the limitations of finger-stick SMBG and thereby help improve patient outcomes. These systems enable increased frequency of glucose measurements and a better characterization of dynamic glucose fluctuations, including episodes of unrealized hypoglycemia. Furthermore, integration of CGMs with automated insulin pumps allows for establishment of a closed-loop “artificial pancreas” system to more closely approximate physiologic insulin delivery and to improve adherence.

Monitoring real-time analyte measurements from a living body via wireless analyte monitoring sensor(s) may provide numerous health and research benefits. There is a need to enhance such analyte monitoring systems via innovations comprising, but not limited to, multiple modes of operation for a transceiver of an analyte monitoring system, which may be particularly useful for meeting the requirements of a clinical trial.

### SUMMARY

Aspects of the present invention may relate to methods and systems for analyte monitoring. More specifically, some aspects of the present invention may relate to multiple modes of operation for a transceiver in an analyte monitoring system.

One aspect of the invention may provide an analyte monitoring system including an analyte sensor, a transceiver, and a display device. The transceiver may be configured to operate in two or more modes of operation. The display device may be configured to communicate with the transceiver. The two or more modes of operation may include a clinical mode in which the transceiver receives first sensor data from the analyte sensor and calculates one or more first analyte levels using at least the received first sensor data but does not convey the one or more first analyte levels to the display device.

In some embodiments, the transceiver operating according to the clinical mode may calculate one or more first analyte level trends using at least the one or more first analyte levels but does not convey the one or more first analyte level trends to the display device.

In some embodiments, the transceiver operating according to the clinical mode may (i) generate one or more of alerts, alarms, and notifications and (ii) convey some but not all of the generated alerts, alarms, or notifications to the display device. In some embodiments, the generated but not conveyed alerts, alarms, or notifications may include analyte-related alerts, alarms, or notifications. In some embodiments, the conveyed alerts, alarms, or notifications may include analyte-unrelated alerts, alarms, or notifications. In some embodiments, the conveyed alerts, alarms, or notifications may include one or more calibration notifications and/or one or more transceiver battery level notifications.

In some embodiments, the two or more modes of operation may include an unblinded mode in which the transceiver receives second sensor data from the analyte sensor, calculates one or more second analyte levels using at least the received second sensor data, and conveys the one or more second analyte levels to the display device. In some embodiments, the transceiver operating according to the unblinded mode may calculate one or more second analyte level trends using at least the one or more second analyte levels and conveys the one or more second analyte level trends to the display device. In some embodiments, the transceiver operating according to the unblinded mode may generate one or more of alerts, alarms, and notifications and convey the generated alerts, alarms, or notifications to the display device. In some embodiments, the conveyed alerts, alarms, or notifications may include analyte-related alerts, alarms, or notifications.

In some embodiments, the two or more modes of operation may include a blinded mode in which the transceiver receives third sensor data from the analyte sensor but does not calculate one or more analyte levels using the received third sensor data. In some embodiments, the transceiver may be configured to (i) receive a mode command that specifies one of the two or more modes of operation from the display device and (ii) operate according to the specified mode of operation. In some embodiments, one or more of the transceiver and the display device may be configured to activate a timer to keep track of how long the transceiver operates according to the clinical mode.

In some embodiments, the display device may be configured to determine in which of the two or more modes of operation the transceiver is operating. In some embodiments, the display device may be configured to determine in which of the two or more modes of operation the transceiver is operating when the display device connects with the transceiver. In some embodiments, the display device may be configured to adapt its operation to the determined mode of transceiver operation. In some embodiments, the display device may be configured to, in response to determining that the transceiver is operating in the clinical mode, not display analyte data.

In some embodiments, the display device may be configured to set the mode in which the transceiver is operating to one of the two or more modes of operation. In some embodiments, the display device may be configured to set the mode in which the transceiver is operating by conveying a command specifying the one of the two or more modes of transceiver operation. In some embodiments, the display device may be configured to set the mode in which the transceiver is operating by modifying the settings of the transceiver. In some embodiments, the display device may be configured to convey a reference measurement, and the transceiver may be configured to receive the reference measurement and use the reference measurement to calibrate calculation of analyte levels based on received sensor data.

Another aspect of the invention may provide a method including operating a transceiver of an analyte monitoring system according to a clinical mode in which the transceiver receives first sensor data from an analyte sensor of the analyte monitoring system and calculates one or more first analyte levels using at least the received first sensor data but does not convey the one or more first analyte levels to a display device of the analyte monitoring system. The method may also include operating the transceiver according to one or more non-clinical modes.

In some embodiments, the one or more non-clinical modes may include an unblinded mode in which the transceiver receives second sensor data from the analyte sensor, calculates one or more second analyte levels using at least the received second sensor data, and conveys the one or more second analyte levels to the display device. In some embodiments, the one or more non-clinical modes may include a blinded mode in which the transceiver receives third sensor data from the analyte sensor but does not calculate one or more analyte levels using the received third sensor data.

In some embodiments, the method may include receiving a mode command that specifies one of the clinical, unblinded, and blinded modes. In some embodiments, the method may include operating the transceiver according to the specified one of the clinical, unblinded, and blinded modes.

In some embodiments, the method may include receiving a mode command that specifies one of the clinical mode and the unblinded mode. In some embodiments, the method may include operating the transceiver according to the specified one of the clinical mode and the unblinded mode.

In some embodiments, operating the transceiver according to the clinical mode may include receiving a reference measurement and using the reference measurement to calibrate calculation of analyte levels based on received sensor data. In some embodiments, operating the transceiver according to the unblinded mode may include receiving a reference measurement and using the reference measurement to calibrate calculation of analyte levels based on received sensor data.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1 is a schematic view illustrating an analyte monitoring system embodying aspects of the present invention.

FIG. 2 is a schematic view illustrating a sensor and transceiver of an analyte monitoring system embodying aspects of the present invention.

FIG. 3 is cross-sectional, perspective view of a transceiver embodying aspects of the invention.

FIG. 4 is an exploded, perspective view of a transceiver embodying aspects of the invention.

FIG. 5 is a schematic view illustrating a transceiver embodying aspects of the present invention.

FIG. 6 illustrates a block diagram of a display device of the analyte monitoring system according to some embodiments.

FIG. 7 illustrates a block diagram of a computer of the display device of the analyte monitoring system according to some embodiments.

FIG. 8 is a flow chart illustrating a process according to some embodiments.

### DETAILED DESCRIPTION OF PREFERRED EMBODIMENTS

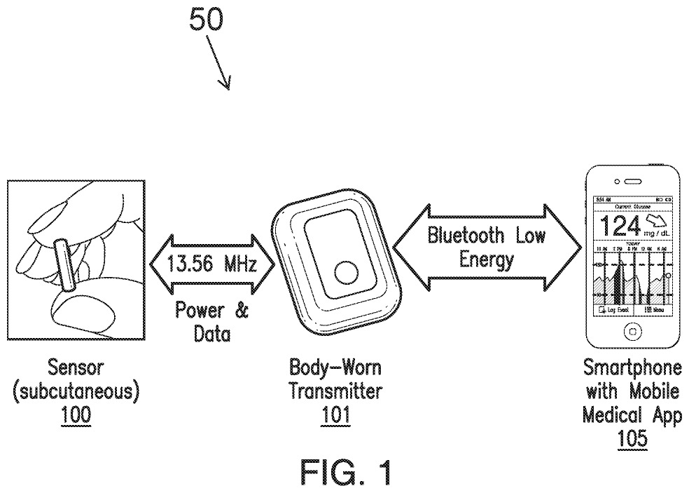

FIG. 1 is a schematic view of an exemplary analyte monitoring system 50 embodying aspects of the present invention. The analyte monitoring system 50 may be a continuous analyte monitoring system (e.g., a continuous glucose monitoring system). In some embodiments, the analyte monitoring system 50 may include one or more of an analyte sensor 100, a transceiver 101, and a display device 105. In some embodiments, the sensor 100 may be small, fully subcutaneously implantable sensor measures analyte (e.g., glucose) concentrations in a medium (e.g., interstitial fluid) of a living animal (e.g., a living human). However, this is not required, and, in some alternative embodiments, the sensor 100 may be a partially implantable (e.g., transcutaneous) sensor or a fully external sensor. In some embodiments, the transceiver 101 may be an externally worn transceiver (e.g., attached via an armband, wristband, waistband, or adhesive patch). In some embodiments, the transceiver 101 may remotely power and/or communicate with the sensor to initiate and receive the measurements (e.g., via near field communication (NFC)). However, this is not required, and, in some alternative embodiments, the transceiver 101 may power and/or communicate with the sensor 100 via one or more wired connections. In some non-limiting embodiments, the transceiver 101 may be a smartphone (e.g., an NFC-enabled smartphone). In some embodiments, the transceiver 101 may communicate information (e.g., one or more analyte concentrations) wirelessly (e.g., via a Bluetooth™ communication standard such as, for example and without limitation Bluetooth Low Energy) to a hand held application running on a display device 105 (e.g., smartphone). In some embodiments, information can be downloaded from the transceiver 101 through a Universal Serial Bus (USB) port. In some embodiments, the analyte monitoring system 50 may include a web interface for plotting and sharing of uploaded data.

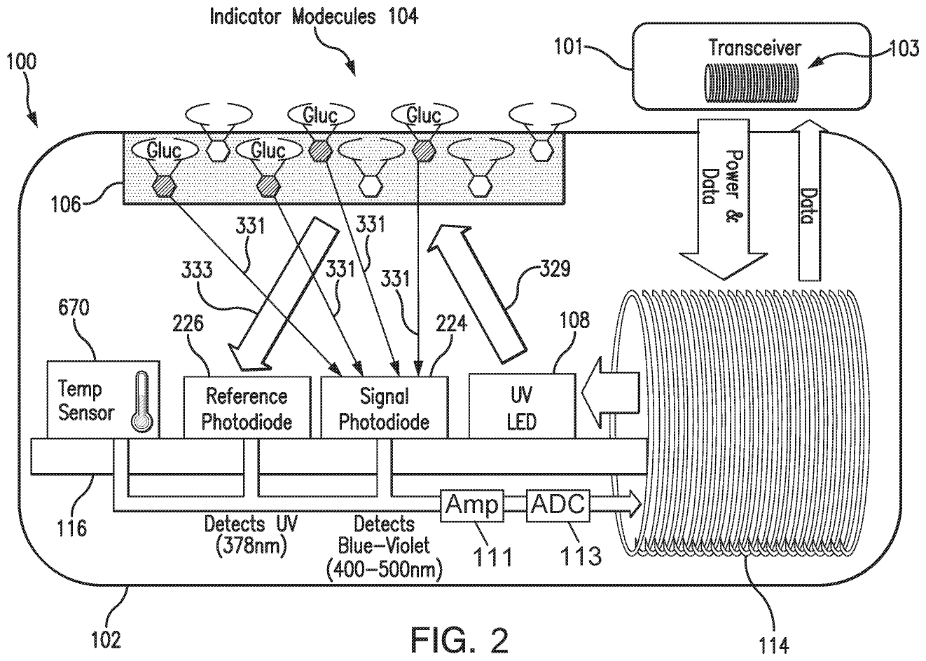

In some embodiments, as illustrated in FIG. 2, the transceiver 101 may include an inductive element 103, such as, for example, a coil. The transceiver 101 may generate an electromagnetic wave or electrodynamic field (e.g., by using a coil) to induce a current in an inductive element 114 of the sensor 100, which powers the sensor 100. The transceiver 101 may also convey data (e.g., commands) to the sensor 100. For example, in a non-limiting embodiment, the transceiver 101 may convey data by modulating the electromagnetic wave used to power the sensor 100 (e.g., by modulating the current flowing through a coil 103 of the transceiver 101). The modulation in the electromagnetic wave generated by the transceiver 101 may be detected/extracted by the sensor 100. Moreover, the transceiver 101 may receive data (e.g., measurement information) from the sensor 100. For example, in a non-limiting embodiment, the transceiver 101 may receive data by detecting modulations in the electromagnetic wave generated by the sensor 100, e.g., by detecting modulations in the current flowing through the coil 103 of the transceiver 101.

The inductive element 103 of the transceiver 101 and the inductive element 114 of the sensor 100 may be in any configuration that permits adequate field strength to be achieved when the two inductive elements are brought within adequate physical proximity.

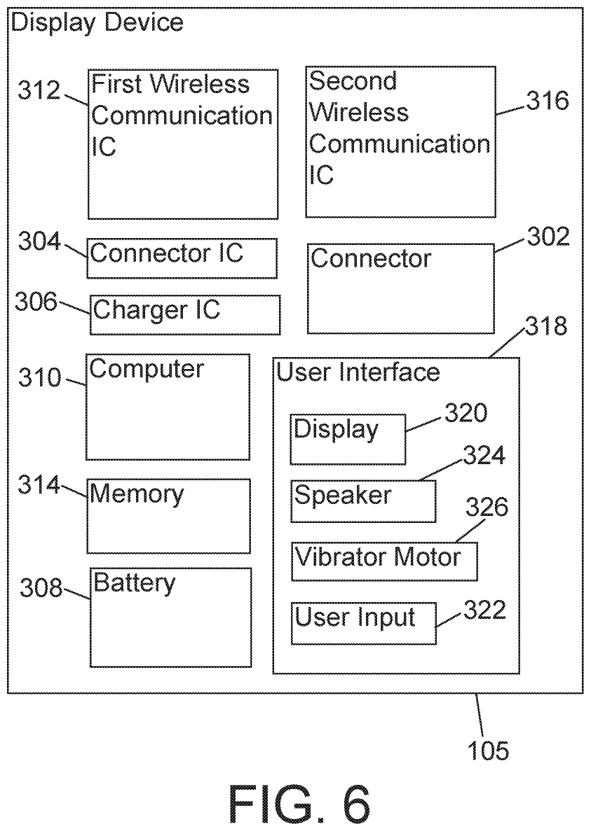

In some non-limiting embodiments, as illustrated in FIG. 2, the sensor 100 may be encased in a sensor housing 102 (i.e., body, shell, capsule, or encasement), which may be rigid and biocompatible. The sensor 100 may include an analyte indicator element 106, such as, for example, a polymer graft coated, diffused, adhered, or embedded on or in at least a portion of the exterior surface of the sensor housing 102. The analyte indicator element 106 (e.g., polymer graft) of the sensor 100 may include indicator molecules 104 (e.g., fluorescent indicator molecules) exhibiting one or more detectable properties (e.g., optical properties) based on the amount or concentration of the analyte in proximity to the analyte indicator element 106. In some embodiments, the sensor 100 may include a light source 108 that emits excitation light 329 over a range of wavelengths that interact with the indicator molecules 104. The sensor 100 may also include one or more photodetectors 224, 226 (e.g., photodiodes, phototransistors, photoresistors, or other photosensitive elements). The one or more photodetectors (e.g., photodetector 224) may be sensitive to emission light 331 (e.g., fluorescent light) emitted by the indicator molecules 104 such that a signal generated by a photodetector (e.g., photodetector 224) in response thereto that is indicative of the level of emission light 331 of the indicator molecules and, thus, the amount of analyte of interest (e.g., glucose). In some non-limiting embodiments, one or more of the photodetectors (e.g., photodetector 226) may be sensitive to excitation light 329 that is reflected from the analyte indicator element 106 as reflection light 333. In some non-limiting embodiments, one or more of the photodetectors may be covered by one or more filters (e.g., bandpass filter 112 of FIG. 6) that allow only a certain subset of wavelengths of light to pass through (e.g., a subset of wavelengths corresponding to emission light 331 or a subset of wavelengths corresponding to reflection light 333) and reflect the remaining wavelengths. In some non-limiting embodiments, the sensor 100 may include a temperature transducer 670. In some non-limiting embodiments, the sensor 100 may include a drug-eluting polymer matrix that disperses one or more therapeutic agents (e.g., an anti-inflammatory drug).

In some embodiments, the outputs of one or more of the photodetectors 224, 226 and the temperature transducer 670 may be amplified by an amplifier 111. In some non-limiting embodiments, the amplifier 111 may be a comparator that receives analog light measurement signals from the photodetectors 224, 226 and output an analog light difference measurement signal indicative of the difference between the received analog light measurement signals. In some non-limiting embodiments, the amplifier 111 may be a transimpedance amplifier. However, in some alternative embodiments, a different amplifier may be used. In some embodiments, the outputs of one or more of the photodetectors 224, 226, the temperature transducer 670, and the amplifier 111 may be converted to a digital signal by an analog-to-digital converter (ADC) 113.

In some embodiments, one or more of the gain of the amplifier 111 and the drive current of the light source 108 may be initially set during a quality control process. In some embodiments, one or more of the gain of the amplifier 111 and the drive current of the light source 108 may be set to allow high dynamic range and to keep the modulated signal within the operational region. In some embodiments, any change (e.g., increase or decrease) to one or more of the drive current of the light source 108 and the gain of the amplifier 111 may change the modulated signal level accordingly.

In some embodiments, as illustrated in FIG. 2, the sensor 100 may include a substrate 116. In some embodiments, the substrate 116 may be a circuit board (e.g., a printed circuit board (PCB) or flexible PCB) on which circuit components (e.g., analog and/or digital circuit components) may be mounted or otherwise attached. However, in some alternative embodiments, the substrate 116 may be a semiconductor substrate having circuitry fabricated therein. The circuitry may include analog and/or digital circuitry. Also, in some semiconductor substrate embodiments, in addition to the circuitry fabricated in the semiconductor substrate, circuitry may be mounted or otherwise attached to the semiconductor substrate 116. In other words, in some semiconductor substrate embodiments, a portion or all of the circuitry, which may include discrete circuit elements, an integrated circuit (e.g., an application specific integrated circuit (ASIC)) and/or other electronic components (e.g., a non-volatile memory), may be fabricated in the semiconductor substrate 116 with the remainder of the circuitry is secured to the semiconductor substrate 116 and/or a core (e.g., ferrite core) for the inductive element 114. In some embodiments, the semiconductor substrate 116 and/or a core may provide communication paths between the various secured components.

In some embodiments, the one or more of the sensor housing 102, analyte indicator element 106, indicator molecules 104, light source 108, photodetectors 224, 226, temperature transducer 670, substrate 116, and inductive element 114 of sensor 100 may include some or all of the features described in one or more of U.S. application Ser. No. 13/761,839, filed on Feb. 7, 2013, U.S. application Ser. No. 13/937,871, filed on Jul. 9, 2013, and U.S. application Ser. No. 13/650,016, filed on Oct. 11, 2012, all of which are incorporated by reference in their entireties. Similarly, the structure and/or function of the sensor 100 and/or transceiver 101 may be as described in one or more of U.S. application Ser. Nos. 13/761,839, 13/937,871, and 13/650,016.

Although in some embodiments, as illustrated in FIG. 2, the sensor 100 may be an optical sensor, this is not required, and, in one or more alternative embodiments, sensor 100 may be a different type of analyte sensor, such as, for example, an electrochemical sensor, a diffusion sensor, or a pressure sensor. Also, although in some embodiments, as illustrated in FIGS. 1 and 2, the analyte sensor 100 may be a fully implantable sensor, this is not required, and, in some alternative embodiments, the sensor 100 may be a transcutaneous sensor having a wired connection to the transceiver 101. For example, in some alternative embodiments, the sensor 100 may be located in or on a transcutaneous needle (e.g., at the tip thereof). In these embodiments, instead of wirelessly communicating using inductive elements 103 and 114, the sensor 100 and transceiver 101 may communicate using one or more wires connected between the transceiver 101 and the transceiver transcutaneous needle that includes the sensor 100. For another example, in some alternative embodiments, the sensor 100 may be located in a catheter (e.g., for intravenous blood glucose monitoring) and may communicate (wirelessly or using wires) with the transceiver 101.

In some embodiments, the sensor 100 may include a transceiver interface device. In some embodiments where the sensor 100 includes an antenna (e.g., inductive element 114), the transceiver interface device may include the antenna (e.g., inductive element 114) of sensor 100. In some of the transcutaneous embodiments where there exists a wired connection between the sensor 100 and the transceiver 101, the transceiver interface device may include the wired connection.

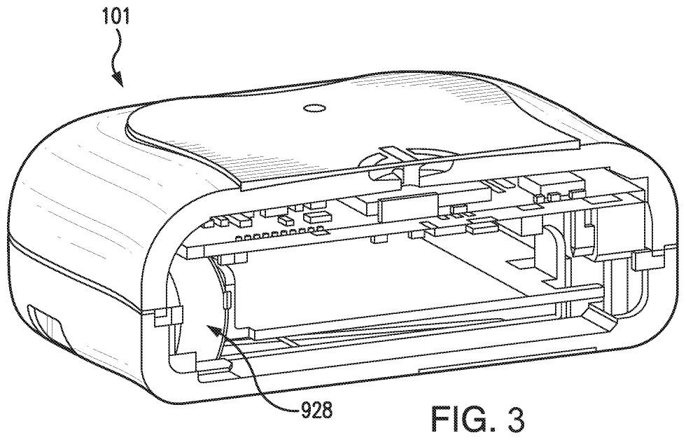

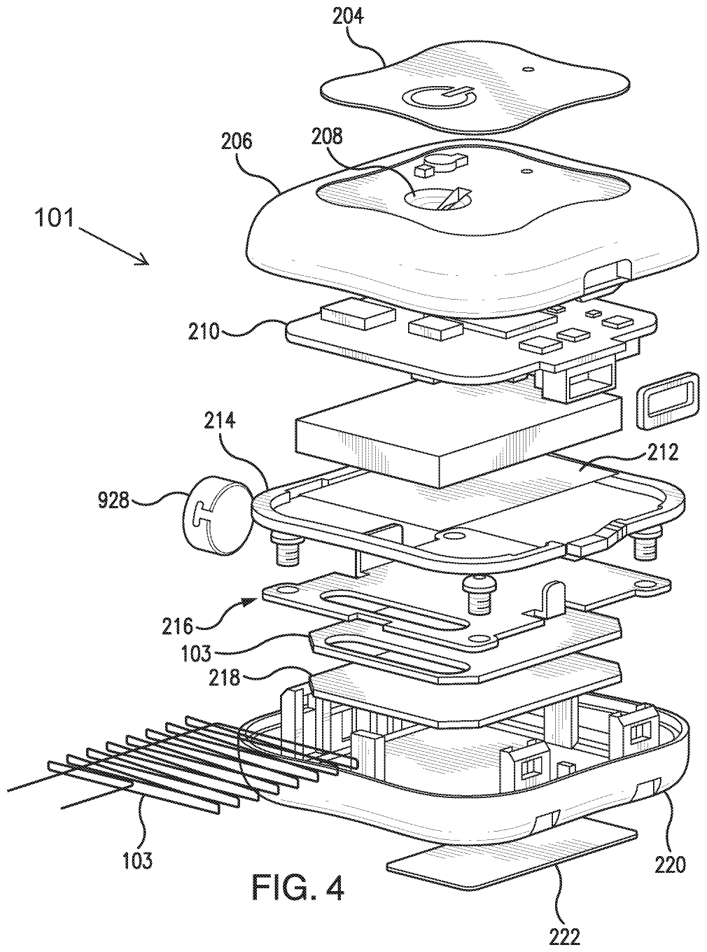

FIGS. 3 and 4 are cross-sectional and exploded views, respectively, of a non-limiting embodiment of the transceiver 101, which may be included in the analyte monitoring system illustrated in FIG. 1. As illustrated in FIG. 4, in some non-limiting embodiments, the transceiver 101 may include a graphic overlay 204, front housing 206, button 208, printed circuit board (PCB) assembly 210, battery 212, gaskets 214, antenna 103, frame 218, reflection plate 216, back housing 220, ID label 222, and/or vibration motor 928. In some non-limiting embodiments, the vibration motor 928 may be attached to the front housing 206 or back housing 220 such that the battery 212 does not dampen the vibration of vibration motor 928. In a non-limiting embodiment, the transceiver electronics may be assembled using standard surface mount device (SMD) reflow and solder techniques. In one embodiment, the electronics and peripherals may be put into a snap together housing design in which the front housing 206 and back housing 220 may be snapped together. In some embodiments, the full assembly process may be performed at a single external electronics house. However, this is not required, and, in alternative embodiments, the transceiver assembly process may be performed at one or more electronics houses, which may be internal, external, or a combination thereof. In some embodiments, the assembled transceiver 101 may be programmed and functionally tested. In some embodiments, assembled transceivers 101 may be packaged into their final shipping containers and be ready for sale.

In some embodiments, as illustrated in FIGS. 3 and 4, the antenna 103 may be contained within the housing 206 and 220 of the transceiver 101. In some embodiments, the antenna 103 in the transceiver 101 may be small and/or flat so that the antenna 103 fits within the housing 206 and 220 of a small, lightweight transceiver 101. In some embodiments, the antenna 103 may be robust and capable of resisting various impacts. In some embodiments, the transceiver 101 may be suitable for placement, for example, on an abdomen area, upper-arm, wrist, or thigh of a patient body. In some non-limiting embodiments, the transceiver 101 may be suitable for attachment to a patient body by means of a biocompatible patch. Although, in some embodiments, the antenna 103 may be contained within the housing 206 and 220 of the transceiver 101, this is not required, and, in some alternative embodiments, a portion or all of the antenna 103 may be located external to the transceiver housing. For example, in some alternative embodiments, antenna 103 may wrap around a user's wrist, arm, leg, or waist such as, for example, the antenna described in U.S. Pat. No. 8,073,548, which is incorporated herein by reference in its entirety.

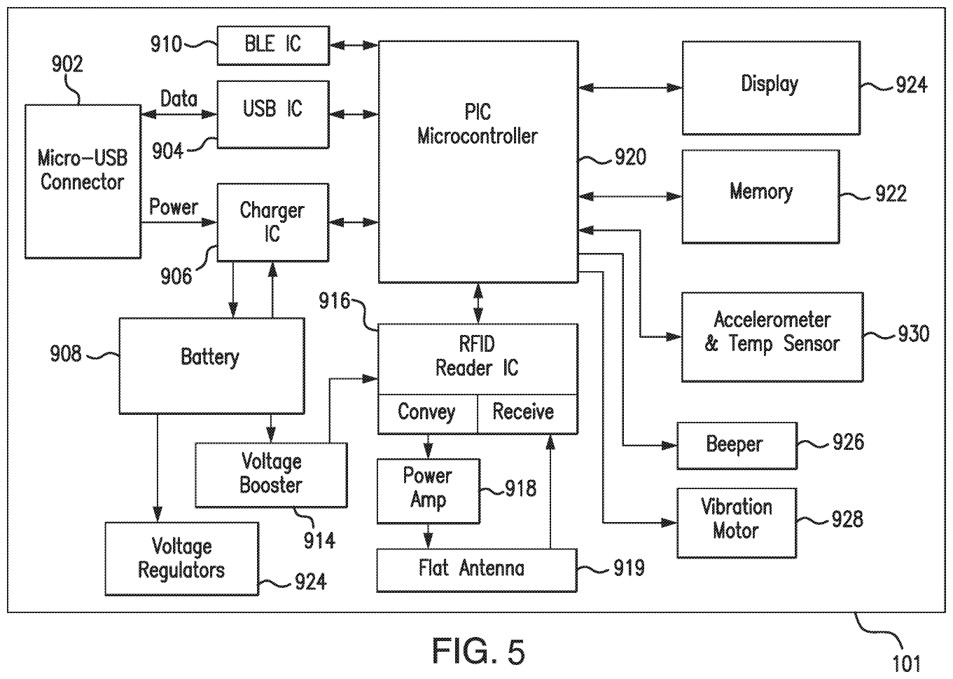

FIG. 5 is a schematic view of an external transceiver 101 according to a non-limiting embodiment. In some embodiments, the transceiver 101 may have a connector 902, such as, for example, a Micro-Universal Serial Bus (USB) connector. The connector 902 may enable a wired connection to an external device, such as a personal computer (e.g., personal computer 109) or a display device 105 (e.g., a smartphone).

The transceiver 101 may exchange data to and from the external device through the connector 902 and/or may receive power through the connector 902. The transceiver 101 may include a connector integrated circuit (IC) 904, such as, for example, a USB-IC, which may control transmission and receipt of data through the connector 902. The transceiver 101 may also include a charger IC 906, which may receive power via the connector 902 and charge a battery 908 (e.g., lithium-polymer battery). In some embodiments, the battery 908 may be rechargeable, may have a short recharge duration, and/or may have a small size.

In some embodiments, the transceiver 101 may include one or more connectors in addition to (or as an alternative to) Micro-USB connector 904. For example, in one alternative embodiment, the transceiver 101 may include a spring-based connector (e.g., Pogo pin connector) in addition to (or as an alternative to) Micro-USB connector 904, and the transceiver 101 may use a connection established via the spring-based connector for wired communication to a personal computer (e.g., personal computer 109) or a display device 105 (e.g., a smartphone) and/or to receive power, which may be used, for example, to charge the battery 908.

In some embodiments, the transceiver 101 may have a wireless communication IC 910, which enables wireless communication with an external device, such as, for example, one or more personal computers (e.g., personal computer 109) or one or more display devices 105 (e.g., a smartphone). In one non-limiting embodiment, the wireless communication IC 910 may employ one or more wireless communication standards to wirelessly transmit data. The wireless communication standard employed may be any suitable wireless communication standard, such as an ANT standard, a Bluetooth standard, or a Bluetooth Low Energy (BLE) standard (e.g., BLE 4.0). In some non-limiting embodiments, the wireless communication IC 910 may be configured to wirelessly transmit data at a frequency greater than 1 gigahertz (e.g., 2.4 or 5 GHz). In some embodiments, the wireless communication IC 910 may include an antenna (e.g., a Bluetooth antenna). In some non-limiting embodiments, the antenna of the wireless communication IC 910 may be entirely contained within the housing (e.g., housing 206 and 220) of the transceiver 101. However, this is not required, and, in alternative embodiments, all or a portion of the antenna of the wireless communication IC 910 may be external to the transceiver housing.

In some embodiments, the transceiver 101 may include a display interface device, which may enable communication by the transceiver 101 with one or more display devices 105. In some embodiments, the display interface device may include the antenna of the wireless communication IC 910 and/or the connector 902. In some non-limiting embodiments, the display interface device may additionally include the wireless communication IC 910 and/or the connector IC 904.

In some embodiments, the transceiver 101 may include voltage regulators 912 and/or a voltage booster 914. The battery 908 may supply power (via voltage booster 914) to radio-frequency identification (RFID) reader IC 916, which uses the inductive element 103 to convey information (e.g., commands) to the sensor 101 and receive information (e.g., measurement information) from the sensor 100. In some non-limiting embodiments, the sensor 100 and transceiver 101 may communicate using near field communication (NFC) (e.g., at a frequency of 13.56 MHz). In the illustrated embodiment, the inductive element 103 is a flat antenna. In some non-limiting embodiments, the antenna may be flexible. However, as noted above, the inductive element 103 of the transceiver 101 may be in any configuration that permits adequate field strength to be achieved when brought within adequate physical proximity to the inductive element 114 of the sensor 100. In some embodiments, the transceiver 101 may include a power amplifier 918 to amplify the signal to be conveyed by the inductive element 103 to the sensor 100.

The transceiver 101 may include a computer 920 and a memory 922 (e.g., Flash memory). In some non-limiting embodiments, the memory 922 may be non-volatile and/or capable of being electronically erased and/or rewritten. In some embodiments, the computer 920 may include a processor and a non-transitory memory. In some non-limiting embodiments, the computer 920 may be, for example and without limitation, a peripheral interface controller (PIC) microcontroller. In some embodiments, the computer 920 may control the overall operation of the transceiver 101. For example, the computer 920 may control the connector IC 904 or wireless communication IC 910 to transmit data via wired or wireless communication and/or control the RFID reader IC 916 to convey data via the inductive element 103. The computer 920 may also control processing of data received via the inductive element 103, connector 902, or wireless communication IC 910.

In some embodiments, the transceiver 101 may include a sensor interface device, which may enable communication by the transceiver 101 with a sensor 100. In some embodiments, the sensor interface device may include the inductive element 103. In some non-limiting embodiments, the sensor interface device may additionally include the RFID reader IC 916 and/or the power amplifier 918. However, in some alternative embodiments where there exists a wired connection between the sensor 100 and the transceiver 101 (e.g., transcutaneous embodiments), the sensor interface device may include the wired connection.

In some embodiments, the transceiver 101 may include a display 924 (e.g., liquid crystal display and/or one or more light emitting diodes), which computer 920 may control to display data (e.g., analyte concentration values). In some embodiments, the transceiver 101 may include a speaker 926 (e.g., a beeper) and/or vibration motor 928, which may be activated, for example, in the event that an alarm condition (e.g., detection of a hypoglycemic or hyperglycemic condition) is met. The transceiver 101 may also include one or more additional sensors 930, which may include an accelerometer and/or temperature sensor, that may be used in the processing performed by the computer 920.

In some embodiments, the transceiver 101 may be a body-worn transceiver that is a rechargeable, external device worn over the sensor implantation or insertion site. The transceiver 101 may supply power to the proximate sensor 100, calculate analyte concentrations from data received from the sensor 100, and/or transmit the calculated analyte concentrations to a display device 105 (see FIG. 1). Power may be supplied to the sensor 100 through an inductive link (e.g., an inductive link of 13.56 MHz). In some embodiments, the transceiver 101 may be placed using an adhesive patch or a specially designed strap or belt. The external transceiver 101 may read measured analyte data from a subcutaneous sensor 100 (e.g., up to a depth of 2 cm or more). The transceiver 101 may periodically (e.g., every 2, 5, or 10 minutes) read sensor data and calculate an analyte concentration and an analyte concentration trend. From this information, the transceiver 101 may also determine if an alert and/or alarm condition exists, which may be signaled to the user (e.g., through vibration by vibration motor 928 and/or an LED of the transceiver's display 924 and/or a display of a display device 105). The information from the transceiver 101 (e.g., calculated analyte concentrations, calculated analyte concentration trends, alerts, alarms, and/or notifications) may be transmitted to a display device 105 (e.g., via Bluetooth Low Energy with Advanced Encryption Standard (AES)-Counter CBC-MAC (CCM) encryption) for display by a mobile medical application (MMA) being executed by the display device 105. In some non-limiting embodiments, the MMA may provide alarms, alerts, and/or notifications in addition to any alerts, alarms, and/or notifications received from the transceiver 101. In one embodiment, the MMA may be configured to provide push notifications. In some embodiments, the transceiver 101 may have a power button (e.g., button 208) to allow the user to turn the device on or off, reset the device, or check the remaining battery life. In some embodiments, the transceiver 101 may have a button, which may be the same button as a power button or an additional button, to suppress one or more user notification signals (e.g., vibration, visual, and/or audible) of the transceiver 101 generated by the transceiver 101 in response to detection of an alert or alarm condition.

In some embodiments, the transceiver 101 of the analyte monitoring system 50 receives raw signals indicative of an amount or concentration of an analyte in proximity to the analyte indicator element 106 of the analyte sensor 100. In some embodiments, the transceiver 101 may receive the raw signals from the sensor 100 periodically (e.g., every 5, 10, or 20 minutes). In some embodiments, the raw signals may include one or more analyte measurements (e.g., one or more measurements indicative of the level of emission light 331 from the indicator molecules 104 as measured by the photodetector 224) and/or one or more temperature measurements (e.g., as measured by the temperature transducer 670). In some embodiments, the transceiver 101 may use the received raw signals to calculate analyte concentration. In some embodiments, the transceiver 100 may store one or more calculated analyte concentrations (e.g., in memory 922). In some embodiments, the transceiver 100 may convey one or more calculated analyte concentrations to the display device 105.

In some embodiments, the analyte monitoring system 50 may calibrate the conversion of raw signals to analyte concentration. In some embodiments, the calibration may be performed approximately periodically (e.g., every 12 or 24 hours). In some embodiments, the calibration may be performed using one or more reference measurements (e.g., one or more self-monitoring blood glucose (SMBG) measurements), which may be entered into the analyte monitoring system 50 using the user interface of the display device 105. In some embodiments, the transceiver 101 may receive the one or more reference measurements from the display device 105 and perform the calibration.

In some embodiments, the transceiver 101 may store multiple modes of transceiver operation (e.g., in the memory 922, which may be a non-volatile memory). In some embodiments, the multiple modes of transceiver operation may include one or more of an unblinded mode, a blinded mode, and a clinical mode. In some embodiments, the multiple modes of operation may be useful during a clinical trial, which may need to be blinded at certain times and unblinded at other times. For example, a clinical trial may include one or more blinded in-clinic sessions and one or more unblinded in-home session. In some embodiments, the multiple modes of operation may be useful for a clinical trial that requires a blinded analyte monitoring system but requires that the transceiver in the system be able to deliver certain alarms, alerts, and/or conditions.

In some embodiments, when the transceiver 101 is operating according to the unblinded mode, the operation of the transceiver 101 may include one or more of receiving sensor data (e.g., raw signals) from the analyte sensor 100, calculating one or more analyte levels (e.g., concentrations) using at least the received sensor data, calculating one or more analyte level trends using at least the one or more calculated analyte levels, generating one or more alerts, alarms, and/or notifications, and conveying the one or more calculated analyte levels, the one or more analyte level trends, and the one or more generated alerts, alarms, and/or notifications to the display device 105. In some embodiments, sensor data may include one or more analyte measurements (e.g., one or more measurements indicative of the level of emission light 331 from the indicator molecules 104 as measured by the photodetector 224) and/or one or more temperature measurements (e.g., as measured by the temperature transducer 670). In some embodiments, generating the one or more alerts, alarms, and/or notifications may include generating one or more alarms (e.g., hypoglycemic and hyperglycemic alarms) using at least the one or more calculated analyte levels (e.g., by comparing the one or more calculated analyte levels to hyperglycemia and/or hypoglycemia thresholds). In some embodiments, generating the one or more alerts, alarms, and/or notifications may additionally or alternatively include generating one or more alerts (e.g., predictive alerts) using at least the one or more calculated analyte levels and the one or more calculated analyte level trends (e.g., by predicting whether the analyte level will cross a either of a hyperglycemia or hypoglycemia threshold within a period of time such as, for example and without limitation, 5, 10, or 20 minutes). In some embodiments, generating the one or more alerts, alarms, and/or notifications may additionally or alternatively include generating one or more notifications (e.g., one or more calibration notifications and/or one or more transceiver battery level notifications).

In some embodiments, when the transceiver 101 is operating according to the blinded mode, the operation of the transceiver 101 may include receiving sensor data (e.g., raw signals) from the analyte sensor 100. However, in some embodiments, in contrast with the unblinded mode, the transceiver 101 operating according to the blinded mode neither calculates one or more analyte levels (e.g., concentrations) nor calculates one or more analyte level trends. In some non-limiting embodiments, the transceiver 101 operating according to the blinded mode does not generate one or more of alerts, alarms, and notifications.

In some embodiments, when the transceiver 101 is operating according to the clinical mode, the operation of the transceiver 101 may include one or more of receiving sensor data (e.g., raw signals) from the analyte sensor 100, calculating one or more analyte levels (e.g., concentrations) using at least the received sensor data, and calculating one or more analyte level trends using at least the one or more calculated analyte levels. However, in some embodiments, in contrast with the unblinded mode, the transceiver 101 operating according to the clinical mode does not convey any of the one or more calculated analyte levels and the one or more calculated analyte level trends to the display device 105. In some embodiments, the transceiver 101 operating according to the clinical mode may generate one or more alerts, alarms, and/or notifications. However, in some embodiments, the transceiver 101 operating according to the clinical mode conveys some but not all of the generated alerts, alarms, and/or notifications to the display device 105. In some non-limiting embodiments, the transceiver 101 operating according to the clinical mode does not convey analyte-related alerts or alarms (e.g., high glucose or low glucose) to the display device 105. In some non-limiting embodiments, the transceiver 101 operating according to the clinical mode may convey notifications to the display device 105 but does not convey any alerts or alarms to the display device 105. In some non-limiting embodiments, the transceiver 101 operating according to the clinical mode conveys one or more calibration notifications and/or one or more transceiver battery level notifications to the display device 105 but does not convey other notifications, alarms, or alerts.

FIG. 6 is a block diagram of a non-limiting embodiment of the display device 105 of the analyte monitoring system 50. As shown in FIG. 6, in some embodiments, the display device 105 may include one or more of a connector 302, a connector integrated circuit (IC) 304, a charger IC 306, a battery 308, a computer 310, a first wireless communication IC 312, a memory 314, a second wireless communication IC 316, and a user interface 318.

In some embodiments in which the display device 105 includes the connector 302, the connector 302 may be, for example and without limitation, a Micro-Universal Serial Bus (USB) connector. The connector 302 may enable a wired connection to an external device, such as a personal computer or transceiver 101. The display device 105 may exchange data to and from the external device through the connector 302 and/or may receive power through the connector 302. In some embodiments, the connector IC 304 may be, for example and without limitation, a USB-IC, which may control transmission and receipt of data through the connector 302.

In some embodiments in which the display device 105 includes the charger IC 306, the charger IC 306 may receive power via the connector 302 and charge the battery 308. In some non-limiting embodiments, the battery 308 may be, for example and without limitation, a lithium-polymer battery. In some embodiments, the battery 308 may be rechargeable, may have a short recharge duration, and/or may have a small size.

In some embodiments, the display device 105 may include one or more connectors and/or one or more connector ICs in addition to (or as an alternative to) connector 302 and connector IC 304. For example, in some alternative embodiments, the display device 105 may include a spring-based connector (e.g., Pogo pin connector) in addition to (or as an alternative to) connector 302, and the display device 105 may use a connection established via the spring-based connector for wired communication to a personal computer or the transceiver 101 and/or to receive power, which may be used, for example, to charge the battery 308.

In some embodiments in which the display device 105 includes the first wireless communication IC 312, the first wireless communication IC 312 may enable wireless communication with one or more external devices, such as, for example, one or more personal computers, one or more transceivers 101, and/or one or more other display devices 105. In some non-limiting embodiments, the first wireless communication IC 312 may employ one or more wireless communication standards to wirelessly transmit data. The wireless communication standard employed may be any suitable wireless communication standard, such as an ANT standard, a Bluetooth standard, or a Bluetooth Low Energy (BLE) standard (e.g., BLE 4.0). In some non-limiting embodiments, the first wireless communication IC 312 may be configured to wirelessly transmit data at a frequency greater than 1 gigahertz (e.g., 2.4 or 5 GHz). In some embodiments, the first wireless communication IC 312 may include an antenna (e.g., a Bluetooth antenna). In some non-limiting embodiments, the antenna of the first wireless communication IC 312 may be entirely contained within a housing of the display device 105. However, this is not required, and, in alternative embodiments, all or a portion of the antenna of the first wireless communication IC 312 may be external to the display device housing.

In some embodiments, the display device 105 may include a transceiver interface device, which may enable communication by the display device 105 with one or more transceivers 101. In some embodiments, the transceiver interface device may include the antenna of the first wireless communication IC 312 and/or the connector 302. In some non-limiting embodiments, the transceiver interface device may additionally or alternatively include the first wireless communication IC 312 and/or the connector IC 304.

In some embodiments in which the display device 105 includes the second wireless communication IC 316, the second wireless communication IC 316 may enable the display device 105 to communicate with one or more remote devices (e.g., smartphones, servers, and/or personal computers) via wireless local area networks (e.g., Wi-Fi), cellular networks, and/or the Internet. In some non-limiting embodiments, the second wireless communication IC 316 may employ one or more wireless communication standards to wirelessly transmit data. In some embodiments, the second wireless communication IC 316 may include one or more antennas (e.g., a Wi-Fi antenna and/or one or more cellular antennas). In some non-limiting embodiments, the one or more antennas of the second wireless communication IC 316 may be entirely contained within a housing of the display device 105. However, this is not required, and, in alternative embodiments, all or a portion of the one or more antennas of the second wireless communication IC 316 may be external to the display device housing.

In some embodiments in which the display device 105 includes the memory 314, the memory 314 may be non-volatile and/or capable of being electronically erased and/or rewritten. In some embodiments, the memory 314 may be, for example and without limitations a Flash memory.

In some embodiments in which the display device 105 includes the computer 310, the computer 310 may control the overall operation of the display device 105. For example, the computer 310 may control the connector IC 304, the first wireless communication IC 312, and/or the second wireless communication IC 316 to transmit data via wired or wireless communication. The computer 310 may additionally or alternatively control processing of received data (e.g., analyte monitoring data received from the transceiver 101).

In some embodiments in which the display device 105 includes the user interface 318, the user interface 318 may include one or more of a display 320 and a user input 322. In some embodiments, the display 320 may be a liquid crystal display (LCD) and/or light emitting diode (LED) display. In some non-limiting embodiments, the user input 322 may include one or more buttons, a keyboard, a keypad, and/or a touchscreen. In some embodiments, the computer 310 may control the display 320 to display data (e.g., analyte concentration values, analyte trend information, alerts, alarms, and/or notifications). In some embodiments, the user interface 318 may include one or more of a speaker 324 (e.g., a beeper) and a vibration motor 326, which may be activated, for example, in the event that a condition (e.g., a hypoglycemic or hyperglycemic condition) is met.

In some embodiments, the computer 310 may execute a mobile medical application (MMA). In some embodiments, the display device 105 may receive analyte monitoring data from the transceiver 101. The received analyte monitoring data may include one or more analyte concentrations, one or more analyte concentrations trends, and/or one or more sensor measurements. The received analyte monitoring data may additionally or alternatively include alarms, alerts, and/or notifications. The MMA may display some or all of the received analyte monitoring data on the display 320 of the display device 105.

In some embodiments, the analyte monitoring system 50 may calibrate the conversion of raw sensor measurements to analyte concentrations. In some embodiments, the calibration may be performed approximately periodically (e.g., every 12 or 24 hours). In some embodiments, the calibration may be performed using one or more reference measurements (e.g., one or more self-monitoring blood glucose (SMBG) measurements). In some embodiments, the reference measurements may be entered into the analyte monitoring system 50 using the user interface 318 of the display device 105. In some embodiments, the display device 105 may convey one or more references measurements to the transceiver 101, and the transceiver 101 may use the one or more received reference measurements to perform the calibration.

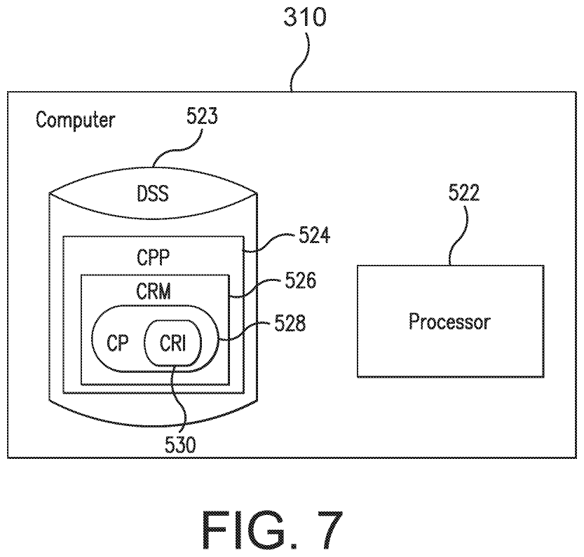

FIG. 7 is a block diagram of a non-limiting embodiment of the computer 310 of the analyte monitoring system 50. As shown in FIG. 7, in some embodiments, the computer 310 may include one or more processors 522 (e.g., a general purpose microprocessor) and/or one or more circuits, such as an application specific integrated circuit (ASIC), field-programmable gate arrays (FPGAs), a logic circuit, and the like. In some embodiments, the computer 310 may include a data storage system (DSS) 523. The DSS 523 may include one or more non-volatile storage devices and/or one or more volatile storage devices (e.g., random access memory (RAM)). In embodiments where the computer 310 includes a processor 522, the DSS 523 may include a computer program product (CPP) 524. CPP 524 may include or be a computer readable medium (CRM) 526. The CRM 526 may store a computer program (CP) 528 comprising computer readable instructions (CRI) 530. In some embodiments, the CRM 526 may store, among other programs, the MMA, and the CRI 530 may include one or more instructions of the MMA. The CRM 526 may be a non-transitory computer readable medium, such as, but not limited, to magnetic media (e.g., a hard disk), optical media (e.g., a DVD), solid state devices (e.g., random access memory (RAM) or flash memory), and the like. In some embodiments, the CRI 530 of computer program 528 may be configured such that when executed by processor 522, the CRI 530 causes the computer 310 to perform steps described below (e.g., steps described below with reference to the MMA). In other embodiments, the computer 310 may be configured to perform steps described herein without the need for a computer program. That is, for example, the computer 310 may consist merely of one or more ASICs. Hence, the features of the embodiments described herein may be implemented in hardware and/or software.

In some embodiments in which the user interface 318 of the display device 105 includes the display 320, the MMA may cause the display device 105 to provide a series of graphical control elements or widgets in the user interface 318, such as a graphical user interface (GUI), shown on the display 320. The MMA may, for example without limitation, cause the display device 105 to display analyte related information in a GUI such as, but not limited to: one or more of analyte information, current analyte concentrations, past analyte concentrations, predicted analyte concentrations, user notifications, analyte status alerts and alarms, trend graphs, arrows, and user-entered events. In some embodiments, the MMA may provide one or more graphical control elements that may allow a user to manipulate aspects of the one or more display screens. Although aspects of the MMA are illustrated and described in the context of glucose monitoring system embodiments, this is not required, and, in some alternative embodiments, the MMA may be employed in other types of analyte monitoring systems.

In some embodiments where the display device 105 communicates with a transceiver 101, which in turn obtains sensor measurement data from the analyte sensor 100, the MMA may cause the display device 105 to receive and display one or more of glucose data, trends, graphs, alarms, and alerts from the transceiver 101. In some embodiments, the MMA may store glucose level history and statistics for a patient on the display device 105 (e.g., in memory 314 and/or DSS 533) and/or in a remote data storage system.

In some embodiments, the MMA being executed by the display device 105 may check the mode of operation in which the transceiver 101 is configured. In some embodiments, the MMA may check the mode of transceiver operation when the MMA connects with the transceiver 101 (e.g., using a BLE protocol). In some embodiments, the MMA may adapt its operation to the mode of operation of the transceiver 101. For example, in some embodiments, if the MMA detects that the transceiver 101 is in clinical mode, the MMA prevent any analyte data (including any analyte data that the MMA may normally pull from the transceiver analyte history logs) from being displayed by the display device 105.

In some embodiments, the MMA being executed on the display device 105 may be configured to set the mode of operation of the transceiver 101. In some embodiments, the MMA may be configured to set the mode of transceiver operation by sending to the transceiver 101 a command to enter a particular mode of operation (e.g., the clinical mode or the unblinded mode). In some embodiments, the MMA may be configured to set the mode of transceiver operation by modifying the settings of the transceiver 101.

In some embodiments, the MMA being executed on the display device 105 may control the transceiver 101 to be in the clinical mode for a fixed amount of time (e.g., 8 hours). In some non-limiting embodiments, the transceiver 101 may be pre-programmed to be in the clinical mode for the fixed amount of time, and the MMA may retrieve information relating to the duration of a clinic session (e.g., when the display device 105 executing the MMA connects with the transceiver 101). In some embodiments, the MMA may additionally or alternatively retrieve information relating to the duration of the clinic session from a remote data management system (e.g., a cloud system), and/or the MMA may receive information relating to the duration of the clinic session that was entered into the display device 105 using the user interface 318. In some embodiments, the MMA may activate and deactivate the clinic session by controlling the mode of operation of the transceiver 101 (e.g., by setting the mode of transceiver operation to the clinical mode and then setting the mode of transceiver operation to the unblinded mode). In some embodiments, the MMA cause the transceiver 101 to operate according to the clinical mode and then activate a timer to countdown (or count up to) the clinic session duration. In some embodiments, after expiration of the clinic session duration, the MMA may cause the transceiver 101 to operate according to the unblinded mode. In some non-limiting embodiments, the transceiver 101 may maintain the countdown (or count up) clock for the clinic session when the transceiver 101 is disconnected from the display device 105. In some embodiments, when the transceiver 101 reconnects with the MMA being executed by the display device 105, the MMA may detect the status of the transceiver countdown clock (e.g., by reading a register setting in the transceiver 101 that indicates the countdown of the clinic session duration) and determine whether the transceiver 101 is still in the clinical mode. In some embodiments, after expiration of the countdown clock, the MMA may then control the transceiver 101 to operate according to a non-clinical mode (e.g., the unblinded mode). However, this is not required, and, in some alternative embodiments, the transceiver 101 may control its mode of operation independent of the MMA executing on the display device 105.

In some embodiments, the analyte monitoring system 50 may additionally or alternatively be configured for use in a clinic session having a variable length (instead of a fixed amount of time). In these embodiments, the MMA may command the transceiver 101 or modify the settings of the transceiver 101 to enable or disable the clinical mode of operation. In some non-limiting embodiments, the clinic session may be programmed on a remote data management system (e.g., cloud server) for each user, and the MMA may pull the information for a particular user (e.g., a user in which the sensor 100 is implanted or inserted) from the remote data management system. In some non-limiting embodiments, the MMA may use information about a clinic session to enable or disable a mode of operation in the transceiver 101 (e.g., if the clinic personnel fails to remember to enable or disable the mode of operation for the clinic).

In some non-limiting embodiments, the clinical mode may be the default mode of operation for the transceiver 101, and the unblinded mode of transceiver operation may occur only in the clinic environment. In some such embodiments, the analyte monitoring system 50 may prevent a user from viewing historical data that had been blinded in the default clinical mode of operation. In some embodiments, the MMA may retrieve the default mode of transceiver operation when the display device 105 connects with the transceiver 101. In some non-limiting embodiments, the default mode of transceiver operation may be set, for example, during manufacturing of the transceiver 101. In some non-limiting embodiments, during a clinic session in which the data is unblinded, the MMA may continue to only pull the analyte data for only the current analyte measurement and not pull any historic data that the transceiver 101 had generated when in the clinical mode. It is not required that the clinical mode be the default mode, and, in some alternative embodiments, the unblinded or blinded mode of operation may be the default mode of operation for the transceiver 101.

In some embodiments, when the transceiver 101 is disconnected from the display device 105 and then reconnected, the MMA being executed on the display device 105 may read clinic session start date and/or start time information from the transceiver 101 and pull historical analyte data from the clinic start date and time to the current date and time.

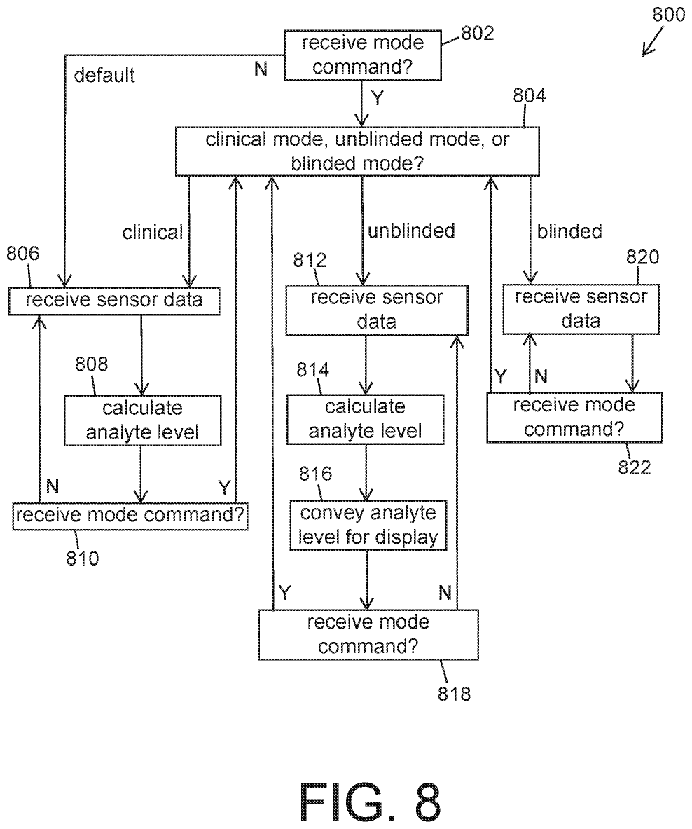

FIG. 8 is a flow chart illustrating a process 800 according to some non-limiting embodiments of the invention. In some embodiments, the transceiver 101 (e.g., the computer 920 of the transceiver 101) may perform one or more steps of the process 800.

In some embodiments, as shown in FIG. 8, the process 800 may include a step 802 in which the transceiver 101 determines whether the transceiver 101 has received a mode command that specifies one of the clinical, unblinded, and blinded modes. In some embodiments, if a mode command has been received, the process 800 may proceed from step 802 to a step 804 in which the transceiver 101 sets one of the clinical, unblinded, and blinded modes according to the specified one of the clinical, unblinded, and blinded modes.

In some embodiments, if the received mode command specified the clinical mode (or if no mode command was received and the clinical mode is the default mode of operation for the transceiver 101), the process 800 may proceed to a step 806 in which the transceiver 101 receives sensor data from the analyte sensor 100 of the analyte monitoring system 50. In some embodiments, the process 800 may include a step 808 in which the transceiver 101 calculates one or more analyte levels using at least the sensor data received in step 806. In some embodiments, the transceiver 101 does not convey the one or more analyte levels calculated in step 808 to the display device 105 of the analyte monitoring system 50. In some embodiments, the process 800 may include a step 810 in which the transceiver 101 determines whether the transceiver 101 has received a mode command that specifies one of the clinical, unblinded, and blinded modes. In some embodiments, if no mode command was received, the transceiver 101 may stay in the clinical mode and proceed back to step 806. In some embodiments, if a mode command was received, the transceiver 101 may proceed back to step 804 in which the transceiver 101 sets one of the clinical, unblinded, and blinded modes according to the newly specified mode.

In some embodiments, if the received mode command specified the unblinded mode (or if no mode command was received and the unblinded mode is the default mode of operation for the transceiver 101), the process 800 may proceed to a step 812 in which the transceiver 101 receives sensor data from the analyte sensor 100 of the analyte monitoring system 50. In some embodiments, the process 800 may include a step 814 in which the transceiver 101 calculates one or more analyte levels using at least the sensor data received in step 812. In some embodiments, the process 800 may include a step 816 of conveying one or more analyte levels calculated in step 814 to the display device 105 of the analyte monitoring system 50. In some embodiments, the process 800 may include a step 816 in which the transceiver 101 determines whether the transceiver 101 has received a mode command that specifies one of the clinical, unblinded, and blinded modes. In some embodiments, if no mode command was received, the transceiver 101 may stay in the unblinded mode and proceed back to step 812. In some embodiments, if a mode command was received, the transceiver 101 may proceed back to step 804 in which the transceiver 101 sets one of the clinical, unblinded, and blinded modes according to the newly specified mode.

In some embodiments, if the received mode command specified the clinical mode (or if no mode command was received and the blinded mode is the default mode of operation for the transceiver 101), the process 800 may proceed to a step 820 in which the transceiver 101 receives sensor data from the analyte sensor 100 of the analyte monitoring system 50. In some embodiments, the transceiver 101 does not calculate one or more analyte levels using the sensor data received in step 820. In some embodiments, the process 800 may include a step 822 in which the transceiver 101 determines whether the transceiver 101 has received a mode command that specifies one of the clinical, unblinded, and blinded modes. In some embodiments, if no mode command was received, the transceiver 101 may stay in the blinded mode and proceed back to step 820. In some embodiments, if a mode command was received, the transceiver 101 may proceed back to step 804 in which the transceiver 101 sets one of the clinical, unblinded, and blinded modes according to the newly specified mode.

Embodiments of the present invention have been fully described above with reference to the drawing figures. Although the invention has been described based upon these preferred embodiments, it would be apparent to those of skill in the art that certain modifications, variations, and alternative constructions could be made to the described embodiments within the spirit and scope of the invention.

What is claimed is:

1. An analyte monitoring system comprising:
an analyte sensor;
a transceiver configured to operate in two or more modes of operation; and
a display device configured to communicate with the transceiver, wherein the display device is configured to (a) determine in which one of the two or more modes of operation the transceiver is operating or (b) set the mode in which the transceiver is operating to one of the two or more modes of operation;
wherein the two or more modes of operation include (i) a clinical mode in which the transceiver receives first sensor data from the analyte sensor and calculates one or more first analyte levels using at least the received first sensor data but does not convey the one or more first analyte levels to the display device and (ii) an unblinded mode in which the transceiver receives second sensor data from the analyte sensor, calculates one or more second analyte levels using at least the received second sensor data, and conveys the one or more second analyte levels to the display device.

2. The system of claim 1, wherein the transceiver operating according to the clinical mode calculates one or more first analyte level trends using at least the one or more first analyte levels but does not convey the one or more first analyte level trends to the display device.

3. The system of claim 1, wherein the transceiver operating according to the clinical mode (i) generates one or more of alerts, alarms, and notifications and (ii) conveys some but not all of the generated alerts, alarms, or notifications to the display device.

4. The system of claim 3, wherein the generated but not conveyed alerts, alarms, or notifications include analyte-related alerts, alarms, or notifications.

5. The system of claim 3, wherein the conveyed alerts, alarms, or notifications include analyte-unrelated alerts, alarms, or notifications.

6. The system of claim 3, wherein the conveyed alerts, alarms, or notifications include one or more calibration notifications and/or one or more transceiver battery level notifications.

7. The system of claim 1, wherein the transceiver operating according to the unblinded mode calculates one or more second analyte level trends using at least the one or more second analyte levels and conveys the one or more second analyte level trends to the display device.

8. The system of claim 1, wherein the transceiver operating according to the unblinded mode generates one or more of alerts, alarms, and notifications and conveys the generated alerts, alarms, or notifications to the display device.

9. The system of claim 8, wherein the conveyed alerts, alarms, or notifications include analyte-related alerts, alarms, or notifications.

10. The system of claim 1, wherein the two or more modes of operation include a blinded mode in which the transceiver receives third sensor data from the analyte sensor but does not calculate one or more analyte levels using the received third sensor data.

11. The system of claim 1, wherein the transceiver is configured to (i) receive a mode command that specifies one of the two or more modes of operation from the display device and (ii) operate according to the specified mode of operation.

12. The system of claim 1, wherein one or more of the transceiver and the display device are configured to activate a timer to keep track of how long the transceiver operates according to the clinical mode.

13. The system of claim 1, wherein the display device is configured to determine in which of the two or more modes of operation the transceiver is operating.

14. The system of claim 13, wherein the display device is configured to determine in which of the two or more modes of operation the transceiver is operating when the display device connects with the transceiver.

15. The system of claim 13, wherein the display device is configured to adapt its operation to the determined mode of transceiver operation.

16. The system of any claim 13, wherein the display device is configured to, in response to determining that the transceiver is operating in the clinical mode, not display analyte data.

17. The system of claim 1, wherein the display device is configured to set the mode in which the transceiver is operating to one of the two or more modes of operation.

18. The system of claim 17, wherein the display device is configured to set the mode in which the transceiver is operating by conveying a command specifying the one of the two or more modes of transceiver operation.

19. The system of claim 17, wherein the display device is configured to set the mode in which the transceiver is operating by modifying the settings of the transceiver.

20. The system of claim 1, wherein the display device is configured to convey a reference measurement, and the transceiver is configured to receive the reference measurement and use the reference measurement to calibrate calculation of analyte levels based on received sensor data.

21. A method comprising:
operating a transceiver of an analyte monitoring system according to a clinical mode in which the transceiver receives first sensor data from an analyte sensor of the analyte monitoring system and calculates one or more first analyte levels using at least the received first sensor data but does not convey the one or more first analyte levels to a display device of the analyte monitoring system; and
operating the transceiver according to an unblinded mode in which the transceiver receives second sensor data from the analyte sensor, calculates one or more second analyte levels using at least the received second sensor data, and conveys the one or more second analyte levels to the display device.

22. The method of claim 21, further comprising operating the transceiver according to a blinded mode in which the transceiver receives third sensor data from the analyte sensor but does not calculate one or more analyte levels using the received third sensor data.

23. The method of claim 22, further comprising:
receiving a mode command that specifies one of the clinical, unblinded, and blinded modes; and
operating the transceiver according to the specified one of the clinical, unblinded, and blinded modes.

24. The method of claim 21, further comprising:
receiving a mode command that specifies one of the clinical mode and the unblinded mode; and
operating the transceiver according to the specified one of the clinical mode and the unblinded mode.

25. The method of claim 21, wherein operating the transceiver according to the clinical mode comprises receiving a reference measurement and using the reference measurement to calibrate calculation of analyte levels based on received sensor data.

26. The method of claim 21, wherein operating the transceiver according to the unblinded mode comprises receiving a reference measurement and using the reference measurement to calibrate calculation of analyte levels based on received sensor data.
